(19) United States
(12) Patent Application Publication
(10) Pub. No.: US 2006/0111560 A1
(43) Pub. Date: May 25, 2006

(54) Process for the preparation of crystalline form II of clarithromycin

(75) Inventors: Bobba Venkata Siva Kumar, Navi Mumbai (IN); Changdev Namdev Raut, Navi Mumbai (IN); Shekhar Bhaskar Bhirud, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/264,835

(22) Filed: Nov. 1, 2005

Related U.S. Application Data
(60) Provisional application No. 60/623,927, filed on Nov. 1, 2004.

(51) Int. Cl. C07H 17/08

(52) U.S. Cl. 536/7.4

(57) ABSTRACT

A process for preparing Form II crystals of clarithromycin is provided comprising (a) treating clarithromycin with a carboxylic acid in an organic solvent to provide a clarithromycin acid salt; and, (b) heating the clarithromycin acid salt at a temperature capable of providing Form II crystals of clarithromycin.

### PRIORITY

This application claims the benefit under 35 U.S.C. §119 to Provisional Application No. 60/623,927, filed Nov. 1, 2004, and entitled “PROCESS FOR THE PREPARATION OF CLARITHROMYCIN”, the contents of which are incorporated by reference herein.

### BACKGROUND OF THE INVENTION

1. Technical Field

The present invention generally relates to an improved process for the preparation of clarithromycin. More specifically, the present invention relates to a process which prepares Form II crystals of clarithromycin.

2. Description of the Related Art

6-O-methylerythromycin A, also known as clarithromycin, is a semi-synthetic macrolide antibiotic related to erythromycin A and exhibits strong antibacterial activity toward a wide range of bacteria. In virtue of its high stability in the acidic environment of the stomach, clarithromycin can be orally administered to treat many infectious diseases, and also to prevent recurrence of an ulcer when used in a combination with other medicines. Clarithromycin exerts its antibacterial action by binding to the 50S ribosomal subunit of susceptible microorganisms resulting in inhibition of protein synthesis. It is active in vitro against a variety of aerobic and anaerobic gram-positive and gram-negative microorganisms as well as most Mycobacterium avium complex (MAC) microorganisms. Clarithromycin is commercially sold under the trade name Biaxin®.

Clarithromycin is known to exist in at least three distinct polymorphic forms, “Form 0”, “Form I” and “Form II”. Form 0 exists as a solvate, which includes a solvent molecule in the crystalline structure. See, e.g., U.S. Pat. No. 5,945,405, herein incorporated by reference. Form I may be prepared by recrystallizing clarithromycin in ethanol and drying at a temperature of less than 70° C. See, e.g., U.S. Pat. No. 5,858,986, herein incorporated by reference. Form II may be prepared by recrystallizing clarithromycin in ethanol and drying at a temperature of greater than 70° C. The polymorphs may be identified using, for example, powder X-ray diffraction spectophotometry, diffraction scanning calorimeter and infrared spectroscopy. Currently, Form II crystals of clarithromycin are in the marketed formulations of clarithromycin due to the improved thermal stability of Form II over the other polymorphic forms.

U.S. Pat. No. 6,444,796, incorporated by reference herein, discloses a process for the preparation of Form II crystals of clarithromycin. The '796 patent discloses treating clarithromycin with formic acid in an organic solvent to provide crystalline clarithromycin formate, and then neutralizing the clarithromycin formate with a base in a mixture of water and a water-miscible solvent to form the Form II crystals of clarithromycin.

### SUMMARY OF THE INVENTION

In accordance with one embodiment of the present invention, a process for the preparation of Form II crystals of clarithromycin is provided, the process comprising:

(a) treating clarithromycin with a carboxylic acid in one or more organic solvents to obtain a crystalline clarithromycin acid salt; and,

(b) heating the clarithromycin acid salt at a temperature capable of providing Form II crystals of clarithromycin.

In accordance with a second embodiment of the present invention, clarithromycin acetate is provided.

The process of the present invention is very simple and utilizes less materials than the process of the prior art to provide pure Form II crystals of clarithromycin in a high yield starting from, e.g., crude clarithromycin.

### BRIEF DESCRIPTION OF THE DRAWING

FIG. 1 is a powder X-ray diffraction pattern of Form II crystals of clarithromycin of Example 1.

### DETAILED DESCRIPTION OF THE PREFERRED EMBODIMENTS

In one aspect of the present invention, Form II crystals of clarithromycin are prepared. Generally, the process of the present invention involves at least two steps: (a) formation of a crystalline clarithromycin acid salt; and (b) formation of Form II crystals of clarithromycin from the crystalline clarithromycin acid salt.

In accordance with step (a) of the process of the present invention, crystalline clarithromycin acid salt is prepared by treating clarithromycin with a carboxylic acid in one or more organic solvents. The clarithromycin used to prepare the acid salt may be of any purifity or of any crude state or crystalline form obtained from a manufacturing process thereof. Representative methods of preparing clarithromycin are described in, for example, U.S. Pat. Nos. 4,331,803; 4,670,549; 4,672,109; 4,990,602; 5,719,272; 5,837,829; and 6,342,590, the contents of which are incorporated by reference herein.

Suitable carboxylic acids employed in the reaction to form the clarithromycin acid salt include, but are not limited to, one or more substituted or unsubstituted C₁ to about C₄₄ saturated or unsaturated carboxylic acid, polycarboxylic acid, e.g., dicarboxylic acids, and preferably one or more substituted or unsubstituted C₃ to about C₃₄ unsaturated or saturated aliphatic carboxylic acid or aliphatic dicarboxylic acid and the like and mixtures thereof. Examples of such carboxylic acids and dicarboxylic acids are maleic acid, itaconic acid, fumaric acid, phthalic acid formic acid, acetic acid, isophthalic acid, terephthalic acid, naphthalenedicarboxylic acid, tartaric acid, oxalic acid, malonic acid, glutaric acid, pimelic acid, suberic acid, glutaconic acid, azelaic acid, sebacic acid, succinic acid, adipic acid, 1,4-cyclohexyl dicarboxylic acid and the like and mixtures thereof. In one embodiment, the carboxylic acid is acetic acid, which will produce a clarithromycin acetate salt. The molar ratio of carboxylic acid to the starting clarithromycin will ordinarily range from about 1:1 to about 5:1.

The organic solvent which may be employed in step (a) of the present invention includes, but is not limited to, a C₁₋₆ alkanol, C₃₋₆ ketone, C₃₋₈ carboxylic ester, C₁₋₆ nitrile, C₄₋₁₀ ether, benzene, benzene substituted with at least one selected from the group consisting of C₁₋₃ alkyl, C₁₋₃ alkoxy, nitro and halogen, C₅₋₁₂ hydrocarbon, C₁₋₄ nitroalkane, aprotic polar solvent, and the like and mixtures thereof. Representative examples of such solvents include, methanol, ethanol, propanol, isopropanol, butanol, isobutanol, tert-butanol, pentanol, hexanol, ethylene glycol, 1,2- or 1,3-propylene glycol, acetone, methyl ethyl ketone, 2-pentanone, 3-pentanone, methyl isobutyl ketone, methyl acetate, ethyl acetate, propyl acetate, isobutyl acetate, methyl propionate, acetonitrile, propionitrile, ethyl ether, isopropyl ether, methyl tert-butyl ether, tetrahydrofuran, dioxane, ethylene glycol dimethyl ether, ethylene glycol diethyl ether, diethylene glycol dimethyl ether, benzene, toluene, xylene, chlorobenzene, nitrobenzene, anisole, pentane, hexane, heptane, cyclohexane, nitromethane, nitroethane, nitropropane, N,N-dimethyl formamide, N,N-dimethyl acetamide, dimethyl sulfoxide, sulfolane, tetrahydrofluran, 1,4-dioxane, ethyl acetate, acetonitrile, N,N-dimethylformamide, dichloromethane and a mixture thereof. In one embodiment, the solvent can be acetone, methyl ethyl ketone, ethyl acetate, isopropyl acetate, acetonitrile, ethanol, isopropanol, tetrahydrofuran, 1,2-dimethoxyethane and mixtures thereof. In another embodiment of the present invention, the solvent is ethyl acetate.

In step (a), the starting clarithromycin is dissolved in one or more organic solvents at a temperature of, for example, room temperature for a time period sufficient to make a solution or suspension. Next, a carboxylic acid, neat or dissolved in an organic solvent, is added to the solution or suspension. The resulting mixture is cooled to a temperature and time period sufficient to precipitate acid salt crystals of clarithromycin. The precipitated crystals can be isolated by conventional techniques, e.g., filtration, and dried in a conventional manner to obtain a crystalline clarithromycin acid salt. If desired, the clarithromycin acid salt may be further purified by conventional techniques, e.g., recrystallization employing a suitable organic solvent.

In step (b) of the process of the present invention, Form II crystals of clarithromycin are prepared by heating the clarithromycin acid salt of step (a) at a temperature capable of producing Form II crystals of clarithromycin. In one embodiment of the present invention, the clarithromycin acid salt is heated at a temperature of about 100° C. to about 120° C. for a time period of about 5 hours to about 10 hours. It is particularly advantageous to heat the clarithromycin acid salt of step (a) under a vacuum to produce the Form II crystals of clarithromycin.

The process of the present invention is very simple and provides substantially pure Form II crystals of clarithromycin, e.g., a purity greater than or equal to about 97%, preferably greater than or equal to about 98%.

The following example is provided to enable one skilled in the art to practice the invention and are merely illustrative of the invention. The example should not be read as limiting the scope of the invention.

### EXAMPLE 1

Step I: Preparation of Clarithromycin Acetate

Clarithromycin (10 g, 77% purity) was suspended in ethyl acetate (300 ml). Acetic acid (2.5 grams) was added to the suspension and the mixture was heated to reflux for about one hour. The suspension was then cooled to a temperature of about 20° C. The resulting crystals were filtered and washed with cold ethyl acetate to provide clarithromycin acetate (8 grams). Purity 91%; Yield 86%.

Clarithromycin acetate (Purity 91%) was further recrystallized from ethyl acetate to provide purified clarithromycin acetate (Purity 98%).

Step II: Preparation of Form II Crystals of Clarithromycin

Clarithromycin acetate (Purity 98%) of step I was dried under vacuum at a temperature of about 120° C. for about four hours to about five hours to provide Form II crystals of clarithromycin. Purity 98%.

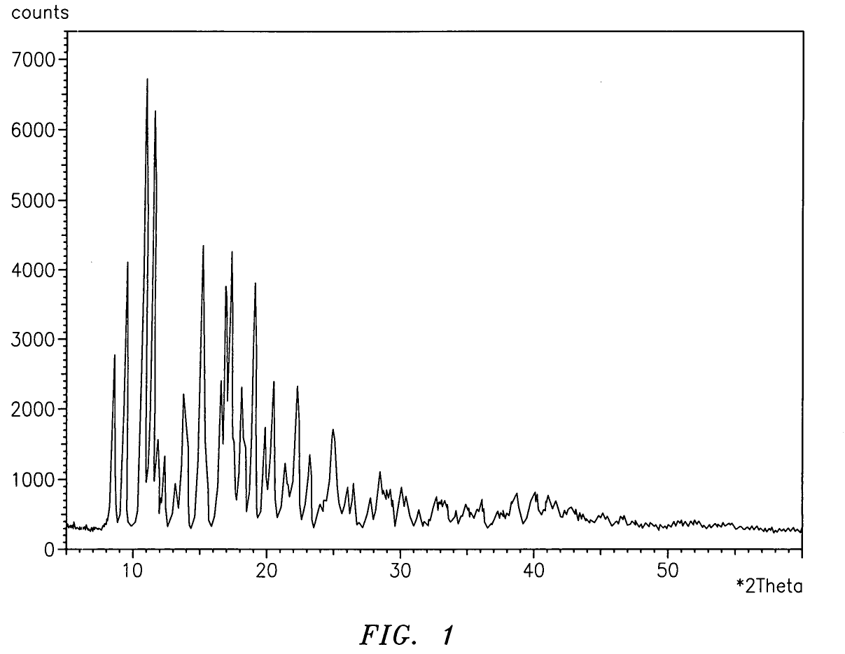

The X-ray Diffraction Pattern of Form II of clarithromycin obtained in the above example is in accordance with FIG. 1.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention.

What is claimed is:

1. A process for preparing Form II crystals of clarithromycin comprising:
(a) treating clarithromycin with a carboxylic acid in one or more organic solvents to provide a clarithromycin acid salt; and,
(b) heating the clarithromycin acid salt at a temperature capable of providing Form II crystals of clarithromycin.

2. The process of claim 1, wherein the carboxylic acid is selected from the group consisting of a substituted or unsubstituted C₁ to about C₄₄ saturated or unsaturated carboxylic acid, substituted or unsubstituted C₁ to about C₄₄ saturated or unsaturated polycarboxylic acid and mixtures thereof.

3. The process of claim 1, wherein the carboxylic acid is selected from the group consisting of a substituted or unsubstituted C₃ to about C₃₄ unsaturated or saturated aliphatic carboxylic acid, substituted or unsubstituted C₃ to about C₃₄ unsaturated or saturated aliphatic dicarboxylic acid and mixtures thereof.

4. The process of claim 1, wherein the carboxylic acid is selected from the group consisting of maleic acid, itaconic acid, fumaric acid, phthalic acid formic acid, acetic acid, isophthalic acid, terephthalic acid, naphthalenedicarboxylic acid, tartaric acid, oxalic acid, malonic acid, glutaric acid, pimelic acid, suberic acid, glutaconic acid, azelaic acid, sebacic acid, succinic acid, adipic acid, 1,4-cyclohexyl dicarboxylic acid and mixtures thereof.

5. The process of claim 1, wherein the carboxylic acid is selected from the group consisting of formic acid, acetic acid, and mixtures thereof.

6. The process of claim 1, wherein the carboxylic acid is acetic acid.

7. The process of claim 1, wherein the molar ratio of the carboxylic acid to clarithromycin is about 1:1 to about 5:1.

8. The process of claim 1, wherein the organic solvent is selected from the group consisting of a C₁₋₆ alkanol, C₃₋₆ ketone, C₃₋₈ carboxylic ester, C₁₋₆ nitrile, C₄₋₁₀ ether, benzene, benzene substituted with at least one selected from the group consisting of C₁₋₃ alkyl, C₁₋₃ alkoxy, nitro and halogen, C₅₋₁₂ hydrocarbon, C₁₋₄ nitroalkane, aprotic polar solvent and mixtures thereof.

9. The process of claim 1, wherein the organic solvent is selected from the group consisting of a methanol, ethanol, propanol, isopropanol, butanol, isobutanol, tert-butanol, pentanol, hexanol, ethylene glycol, 1,2- or 1,3-propylene glycol, acetone, methyl ethyl ketone, 2-pentanone, 3-pentanone, methyl isobutyl ketone, methyl acetate, ethyl acetate, propyl acetate, isobutyl acetate, methyl propionate, acetonitrile, propionitrile, ethyl ether, isopropyl ether, methyl tert-butyl ether, tetrahydrofuran, dioxane, ethylene glycol dimethyl ether, ethylene glycol diethyl ether, diethylene glycol dimethyl ether, benzene, toluene, xylene, chlorobenzene, nitrobenzene, anisole, pentane, hexane, heptane, cyclohexane, nitromethane, nitroethane, nitropropane, N,N-dimethyl formamide, N,N-dimethyl acetamide, dimethyl sulfoxide, sulfolane, tetrahydrofluran, 1,4-dioxane, ethyl acetate, acetonitrile, N,N-dimethylformamide, dichloromethane and mixtures thereof.

10. The process of claim 1, wherein the organic solvent is selected from the group consisting of acetone, methyl ethyl ketone, ethyl acetate, isopropyl acetate, acetonitrile, ethanol, isopropanol, tetrahydrofuran, 1,2-dimethoxyethane and mixtures thereof.

11. The process of claim 1, wherein the organic solvent is ethyl acetate.

12. The process of claim 1, wherein the carboxylic acid is acetic acid and the organic solvent is selected from the group consisting of acetone, methyl ethyl ketone, ethyl acetate, isopropyl acetate, acetonitrile, ethanol, isopropanol, tetrahydrofuran, 1,2-dimethoxyethane and mixtures thereof.

13. The process of claim 1, wherein the carboxylic acid is acetic acid and the organic solvent is ethyl acetate.

14. The process of claim 1, wherein the clarithromycin acid salt is further purified prior to step (b).

15. The process of claim 14, wherein the step of further purifying the clarithromycin acid salt comprise recrystallizing the clarithromycin acid salt in one or more organic solvents.

16. The process of claim 1, wherein the reaction in step (a) is carried out at reflux.

17. The process of claim 1, wherein the temperature maintained in step (b) is about 100° C. to about 120° C.

18. The process of claim 1, wherein step (b) is carried out for a time period of about 5 hours to about 10 hours.

19. The process of claim 1, wherein in step (b) the clarithromycin acid salt is heated under vacuum.

20. The process of claim 1, wherein the Form II crystals of clarithromycin have a purity of greater than or equal to about 97%.

21. The process of claim 1, wherein Form II crystals of clarithromycin have a purity of greater than or equal to about 98%.

22. A compound which is clarithromycin acetate.

23. Clarithromycin acetate having a purity greater than or equal to about 98%.

24. A process for preparing clarithromycin acetate comprising treating clarithromycin with acetic acid in ethyl acetate.

25. The process of claim 24, further comprising recrystallizing clarithromycin acetate in ethyl acetate to provide substantially pure clarithromycin acetate.
